(19) United States
(12) Patent Application Publication
(10) Pub. No.: US 2006/0270977 A1
(43) Pub. Date: Nov. 30, 2006

(54) Rapid exchange balloon catheter with reinforced shaft

(75) Inventors: Beau M. Fisher, Danville, CA (US); Hung Van Vo, Elk Grove, CA (US)

Correspondence Address: CINDY A. LYNCH;CONOR MEDSYSTEMS, INC., 1003 HAMILTON COURT, MENLO PARK, CA, 94025, US

(73) Assignee: Conor Medsystems, Inc., Menlo Park, CA (US)

(21) Appl. No.: 11/441,413

(22) Filed: May 25, 2006

Related U.S. Application Data
(60) Provisional application No. 60/684,775, filed on May 26, 2005.

(51) Int. Cl. A61M 29/00

(52) U.S. Cl. 604/103.04

(57) ABSTRACT

A balloon catheter includes a catheter shaft, a balloon, and a guidewire lumen extending through the lumen. A core wire with a distal coil are positioned in the catheter shaft proximal of the balloon, and a hypotube is positioned in a longitudinally extending inflation lumen, to provide both flexibility and pushability to the catheter shaft.

### CROSS REFERENCE TO RELATED APPLICATIONS

This application claims priority to U.S. Provisional Patent Application Ser. No. 60/684,775, filed May 26, 2005 the entire contents of which are incorporated herein by reference.

### FIELD OF THE INVENTION

The present invention relates to devices, systems, and processes useful as catheters, and more specifically to catheters that can be useful as rapid exchange catheters.

### BRIEF DESCRIPTION OF THE RELATED ART

Rapid exchange balloon catheters are described in U.S. Pat. Nos. 4,762,129 and 5,040,548, the entireties of which are incorporated herein by reference. These rapid exchange catheters include a distal guidewire lumen which extends through the balloon from a distal end of the balloon to a guidewire exit port proximal of the balloon but distal to the proximal end of the catheter. In these and other known rapid exchange balloon catheter systems the catheter shafts include a proximal stiff catheter section extending along about 75% of the catheter length and a distal more flexible portion of the catheter between the stiff section and the balloon. For catheters which are designed for angioplasty or for stent delivery, the portion of the catheter proximal of the balloon and distal to the stiffer proximal catheter section should be simultaneously very flexible to navigate the coronary arteries, have good column strength to provide pushability, and have good kink resistance. The proximal catheter section generally requires good column strength and less flexibility.

Hypotubes or small metal tubes have been used for the proximal sections of rapid exchange catheters due to their excellent pushability and small wall thickness. Braided catheters can also been used for improved kink resistance.

### SUMMARY OF THE INVENTION

According to a first aspect of the invention, a balloon catheter includes a ballon segment having an expandable balloon and a guidewire tube extending through the balloon, the guidewire tube having a proximal port near a proximal end of the balloon and a distal port near a distal end of the balloon, a shaft segment connected to a proximal end of the balloon segment, the shaft segment including an inflation lumen extending from a proximal end of the distal segment to the interior of the balloon segment, and a first stiffening member positioned within a proximal portion of the inflation lumen.

In accordance with a further aspect of the present invention, a balloon catheter is comprised of a balloon segment having an expandable balloon and a guidewire tube extending through the balloon, the guidewire tube having a proximal port proximal of the balloon and distal of a proximal end of the catheter, and a distal port distal of the balloon, a shaft segment connected to a proximal end of the balloon segment, the shaft segment including an inflation lumen having a half-moon-shape, the inflation lumen extending from a proximal end of the distal segment to the interior of the balloon segment, and a hypotube positioned within a proximal portion of the inflation lumen.

In accordance with yet a further aspect of the present invention, a rapid exchange balloon catheter is comprised of a balloon segment having an expandable balloon having a proximal end and a distal end, a shaft segment connected to the proximal end of the balloon segment, the shaft segment including an inflation lumen extending from a proximal end of the distal shaft to the interior of the balloon segment, a guidewire tube extending through the balloon, the guidewire tube having a proximal port near the proximal end of the balloon segment and a distal port near the distal end of the balloon segment, wherein the distal guidewire port is spaced from the balloon segment a distance less than the proximal guidewire port is spaced from the balloon segment, and a metal stiffening member positioned within a proximal portion of the inflation lumen without extending across the proximal port.

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawings.

FIG. 1 illustrates a side perspective view of a catheter system.

FIG. 2 illustrates an enlarged perspective view of portions of the catheter system illustrated in FIG. 1.

FIGS. 3A- 3D illustrate transverse cross-sectional views of the device illustrated in FIG. 2, taken at lines A-A, B-B, C-C, and D-D, respectively.

FIG. 3E illustrates a transverse cross-sectional view of the device illustrated in FIG. 2, taken at lines D-D, according to an alternative embodiment.

FIG. 4 illustrates an enlarged longitudinal cross-sectional view of the device illustrated in FIG. 2, taken immediately proximal of the balloon thereof.

### DETAILED DESCRIPTION OF EXEMPLARY EMBODIMENTS

Referring to the drawing figures, like reference numerals designate identical or corresponding elements throughout the several figures.

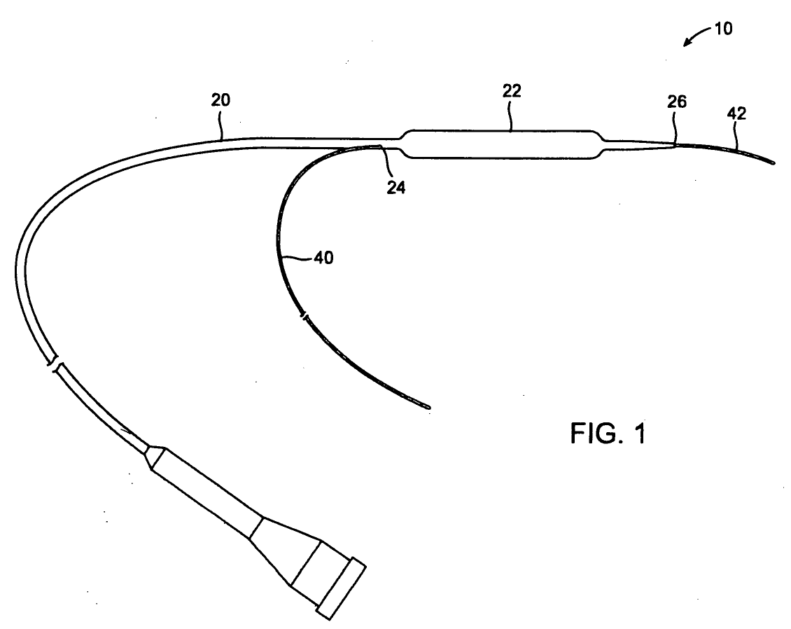

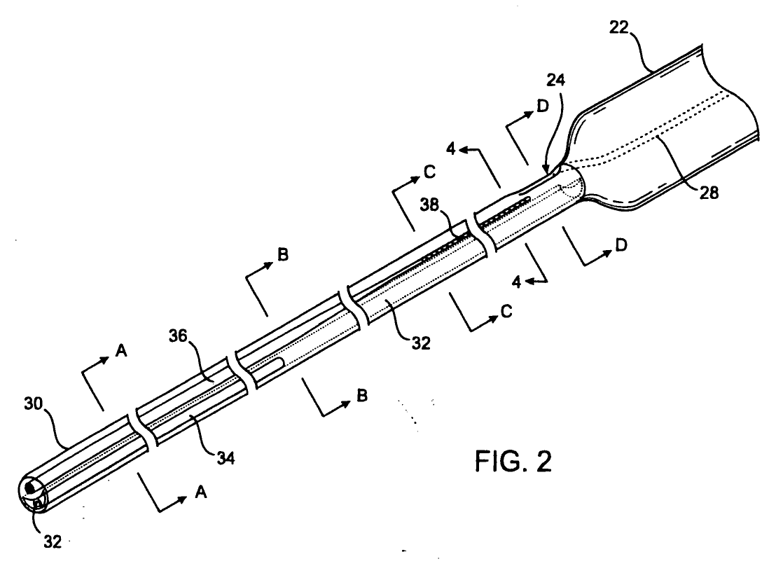

A catheter system 10 illustrated in FIG. 1 embodies principles of the present invention, and includes a catheter 20 (shown schematically), which is optionally a rapid exchange balloon catheter, and a guidewire 40. FIG. 1 illustrates the catheter and balloon in an expanded or inflated configuration. In an unexpanded or delivery configuration, the balloon and stent will have a diameter close to the diameter of the catheter shaft. As illustrated in FIG. 1, the catheter 20 includes a distal guidewire port 26 at a distal end of the catheter and a proximal guidewire port 24 proximal of a balloon 22. According to an exemplary use, the catheter 20 is inserted into a patient, e.g., into the vasculature of a patient, over the guidewire 40 by passing the guidewire into the distal port 26 of the catheter, through a guidewire lumen 28 (see FIGS. 2-4) within the balloon 22, and out the side opening of the proximal guidewire port 24 of the catheter, optionally leaving distal portions 42 of the guidewire extending distally of the catheter.

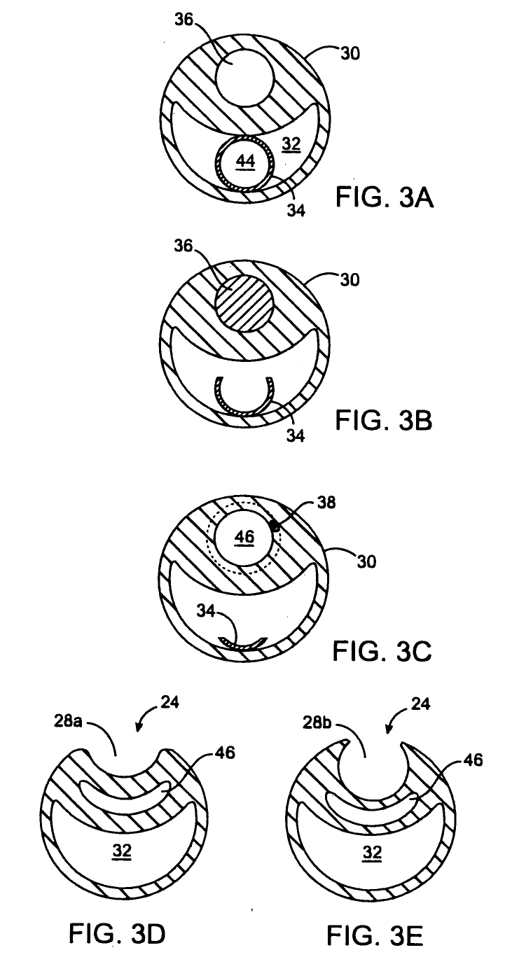

FIG. 2 illustrates an enlarged perspective view of portions of the catheter system illustrated in FIG. 1, embodying principles in accordance with the present invention. The catheter 20 includes a catheter shaft 30 having an inflation lumen 32, a first stiffening member 34, and a second stiffening member 36. One exemplary aspect of the present invention includes that the inflation lumen 32 is generally half-moon-shaped, such as that illustrated generally in FIGS. 2-3E. Other exemplary aspects of the present invention include that the first stiffening member 34 is positioned in the inflation lumen 32 and is formed as a longitudinally extending tube formed of a material so that the stiffening member 34 stiffens proximal portions of the catheter 20. Further exemplary aspects of the present invention include that the first stiffening member 34 can be made of hypotube, and can optionally be tapered, skived, coiled, or notched in distal portions thereof so that the stiffness of the stiffening member 34 decreases distally. When the stiffening member 34 is formed as a tube, the lumen 44 thereof (see FIG. 3A) can optionally be used to conduct inflation fluid to and/or from the balloon 22, that is, to work in conjunction with the half moon shaped inflation lumen 32. The inflation lumen 32 leads from proximal portions of the catheter system 10, through the catheter shaft 30, and to the interior of the balloon 22, as well understood by those of ordinary skill in the art. While FIG. 2 illustrates the stiffening member 34 having a blunt distal end, other embodiments do not have a blunt distal end as described in greater detail elsewhere herein.

FIG. 2 also illustrates other exemplary aspects of the present invention. The guidewire lumen 28 extends through the balloon 22 in a known manner from a proximal port 24. The second stiffening member 36 extends distally from proximal portions of the catheter system 10, through the catheter shaft 30, and terminates proximally of the balloon 22. Another exemplary aspect of the present invention includes that the second stiffening member 36 includes a core wire embedded in the catheter shaft 30, and extends longitudinally along the catheter shaft, generally in parallel to and laterally offset from the inflation lumen 32. Yet another exemplary aspect of the present invention includes that the flexibility of the second stiffening member 36 decreases distally, so that distal portions of the stiffening member 36, e.g., the core wire, are less stiff than proximal portions thereof. A further exemplary aspect of the present invention includes that the second stiffening member 36 includes a coil 38 positioned within the catheter shaft 30 proximal of the balloon 22 and of the proximal port 24 of the guidewire lumen 28. The coil 38 can add both resiliency to the catheter shaft 30 and enhance the flexibility of the shaft proximal of the balloon 22 and the port 24.

For ease of description only, the first stiffening member 34 will be referred to as a hypotube 34, the second stiffening member 36 will be referred to as a core wire 36, and the coil 38 will be referred to separately from the core wire 36.

Turning now to FIGS. 3A-3E, transverse cross-sectional views of the device illustrated in FIG. 2, taken at lines A-A, B-B, C-C, and D-D, respectively, are illustrated. FIGS. 3A-3E illustrate the core wire 36 inside the catheter shaft 30, the inflation lumen 32 having being generally half-moon-shaped, the hypotube 34 extending through the inflation lumen, and the lumen 44 of the hypotube. As discussed elsewhere herein, the interior lumen 44 of the hypotube 34 and the inflation lumen 32 can be used for conducting inflation fluid distally through the catheter shaft 30 to the balloon 22, exhausting any fluid, e.g., air that may be present in the balloon prior to inflation, or combinations thereof, by connecting proximal portions of the two lumens to appropriate inflation devices and/or ports (not illustrated).

FIGS. 3A-3C also illustrate an exemplary aspect of the present invention, discussed briefly elsewhere herein, that the cross-sectional shape of the hypotube 34 can be reduced in more distal portions of the hypotube, e.g., by skiving, forming a coil from the hypotube, or tapering the hypotube. In this manner, the flexibility of the hypotube 34 increases as more material is removed, permitting the catheter system 10 to be more flexible in distal portions. Alternatively, the hypotube 34 can be notched instead of skived, so that the hypotube has a more step-wise reduction in rigidity.

FIG. 3C illustrates the coil 38 positioned in the catheter shaft 30 surrounding a short lumen 46. While FIG. 3C illustrates the coil 38 embedded in the wall of the catheter 30 with a lumen-facing interior surface of the coil exposed within the lumen 46, other aspects of the present invention include that the coil may be fully encapsulated in the catheter material (not illustrated) or the coil may be fully positioned within the lumen 46 and not embedded in the material that makes up the catheter shaft 30.

FIG. 3D illustrates the proximal guidewire exit port 24 and a guidewire channel 28a which is an extension of the guidewire lumen 28. The guidewire channel 28a has a generally open, groove-like configuration, which allows t he guidewire to track alongside of the catheter proximal of the proximal guidewire exit port 24. The lumen 46, at the position of the cross-section of FIG. 3D, is preferably empty and compressed, which assists in making room for the guidewire lumen 28 although the lumen 46 is shown to be still partially open in the compressed configuration, it may also be completely closed. FIG. 3E, which is a cross-sectional view taken at the same position as that for FIG. 3D, illustrates an alternative embodiment in accordance with principles of the present invention, in which the proximal port 24 has a guidewire channel 28b configured more as a semi-closed slot, with a side slot shaped opening that is smaller than the diameter of the guidewire lumen 28.

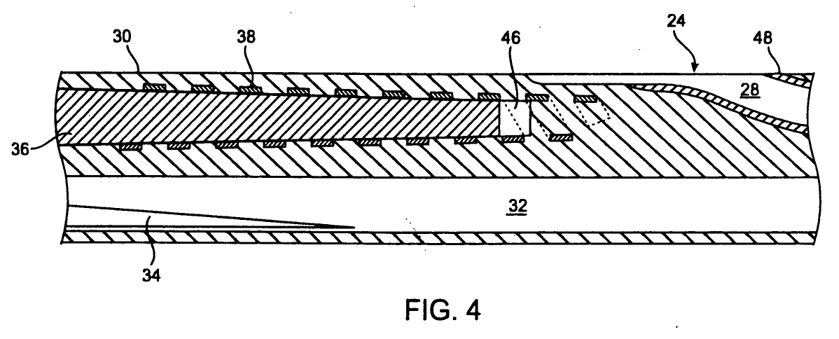

FIG. 4 illustrates an enlarged longitudinal cross-sectional view of the device illustrated in FIG. 2, taken immediately proximal of the balloon thereof. As illustrated in the figure, the hypotube 34 and the core wire 36 preferably taper along more distal portions, as discussed above, in order to permit the catheter shaft 30 to be more flexible in portions proximal of the port 24. Correspondingly, the coil 38 tapers down in cross-sectional diameter. According to the exemplary embodiment illustrated in FIGS. 3C-4, the catheter shaft 30 optionally includes a short distal portion of a lumen 46 in which the core wire 36 is positioned; according to further aspects of the present invention, the distal end of the core wire can be embedding in the catheter shaft 30, eliminating the portion of lumen 46 distal to the core wire. The coil 38 generally surrounds distal portions of the core wire 36, which according to certain aspects of the present invention can be tapered to create a very thin distal end of the core wire. FIG. 4 also illustrates a guidewire lumen shaft 48 ending at its proximal end adjacent to the port 24, and forming the guidewire lumen 28.

Without being limited to particular dimensions, materials, and configurations, in the catheter system 10 it is preferable that: the proximal port 24 is positioned less than about 2 cm from the balloon 22, the proximal port is configured as a groove approximately 3 cm to about 5 cm in length; the hypotube 34 and/or the core wire 36 are secured, e.g., glued, solvent bonded, or heat-staked, into the lumen 46; the hypotube diameter, core wire diameter, inner diameter of lumen 46, inner diameter of lumen 32, and/or outer diameter of the catheter shaft 30 begin to taper down at a position about 100 cm from the proximal end of the catheter shaft; the coil 38 is formed of a metal or a polymer; and/or the outer diameter of the catheter shaft 30 is about 0.05 inches or less, preferably about 0.043 inches or less, before any tapering. The tapering of the lumen 46 and/or the lumen 32, which preferably corresponds with the tapering of the core wire 36 and the hypotube 34, permits the core wire and hypotube to push against the lumen, respectively, which in turn can improve the pushability of the catheter system 10. As will be readily appreciated, the catheter system 10 is constructed of materials that are biocompatible, including but not limited to polymers and certain metals and metal alloys, has surfaces that are atraumatic when inserted through the vasculature of a patient, and are sized to pass through the particular body lumens through which the medical practitioner is to guide it. As such details are not necessary for an appreciation of the principles of the present invention by the ordinarily skilled artisan, they are not included herein so as to not obscure the present invention.

The catheter system 10 of the present invention can be used as a stent delivery system, a dilation catheter, drug delivery catheter, or other catheter. In addition, the catheter described and shown herein has a distal guidewire opening 24 spaced from the balloon 22 a distance less than the proximal guidewire opening 26 is spaced from the balloon. This allows for a short exchange length improving exchange time and will minimize patient blood loss during the wire exchange process. Other exchange lengths may also be used with the invention if desired.

While the invention has been described in detail with reference to exemplary embodiments thereof, it will be apparent to one skilled in the art that various changes can be made, and equivalents employed, without departing from the scope of the invention.

What is claimed is:

1. A balloon catheter comprising:
a balloon segment having an expandable balloon and a guidewire tube extending through the balloon, the guidewire tube having a proximal port near a proximal end of the balloon and a distal port near a distal end of the balloon;
a shaft segment connected to a proximal end of the balloon segment, the shaft segment including an inflation lumen extending from a proximal end of the shaft segment to the interior of the balloon segment; and
a first stiffening member positioned within a proximal portion of the inflation lumen.

2. A balloon catheter according to claim 1, wherein the first stiffening member comprises a hypotube.

3. A balloon catheter according to claim 2, wherein the hypotube has a distal end and the stiffness of the hypotube decreases toward said distal end.

4. A balloon catheter according to claim 2, wherein the hypotube distal end has a shaped selected from the group consisting of skived, coiled, and notched.

5. A balloon catheter according to claim 1, wherein the first stiffening member comprises a longitudinally extending lumen in fluid communication with said inflation lumen.

6. A balloon catheter according to claim 1, wherein the first stiffening member has a distal end proximal of and proximate to said balloon segment.

7. A balloon catheter according to claim 1, wherein the inflation lumen has a cross-section that is half-moon-shaped.

8. A balloon catheter according to claim 1, further comprising:
a second stiffening member extending longitudinally within said catheter segment and parallel to said inflation lumen.

9. A balloon catheter according to claim 8, wherein said second stiffening member comprises a core wire.

10. A balloon catheter according to claim 8, wherein said second stiffening member comprises a coil.

11. A balloon catheter according to claim 8, wherein said second stiffening member comprises a core wire and a coil.

12. A balloon catheter according to claim 11, wherein the core wire comprises a proximal portion, the coil comprises a proximal portion, and the core wire proximal portion is proximal of said coil proximal portion.

13. A balloon catheter according to claim 11, wherein said coil is at least in part positioned around said core wire.

14. A balloon catheter according to claim 11, wherein said coil comprises a distal end, said core wire comprises a distal end, and said coil distal end is distal of said core wire distal end.

15. A balloon catheter according to claim 11, wherein said coil comprises a distal end, said core wire comprises a distal end, and both said coil distal end and said core wire distal end are proximal of said balloon segment.

16. A balloon catheter according to claim 11, wherein said coil is embedded in said shaft segment.

17. A balloon catheter according to claim 11, wherein said core wire is embedded in said shaft segment.

18. A balloon catheter according to claim 8, further comprising:
a second lumen extending longitudinally along said shaft segment to a distal end proximal of said balloon segment;
wherein said second stiffening member comprises a distal end proximal of said second lumen distal end.

19. A balloon catheter according to claim 8, wherein said second stiffening member comprises a coil surrounding said second lumen.

20. A method of using a balloon catheter, comprising:
providing a catheter according to claim 1; and
inflating said balloon segment at least in part by passing fluid through said first stiffening member.

21. A balloon catheter according to claim 7, wherein the first stiffening member is a hypotube.

22. A balloon catheter according to claim 21, wherein the hypotube distal end has a shaped selected from the group consisting of skived, coiled, and notched.

23. A balloon catheter according to claim 21, wherein hypotube is arranged in the inflation lumen to allow inflation fluid to pass through the half-moon-shaped inflation lumen both inside and outside of the hypotube.

24. A balloon catheter according to claim 8, wherein the second stiffening member is positioned in a secondary lumen, and wherein the secondary lumen has a distal end which is compressed to form an exterior longitudinal groove at the proximal guidewire port.

25. A balloon catheter comprising:
a balloon segment having an expandable balloon and a guidewire tube extending through the balloon, the guidewire tube having a proximal port proximal of the balloon and distal of a proximal end of the catheter, and a distal port distal of the balloon;
a shaft segment connected to a proximal end of the balloon segment, the shaft segment including an inflation lumen having a half-moon-shape, the inflation lumen extending from a proximal end of the distal segment to the interior of the balloon segment; and
a hypotube positioned within a proximal portion of the inflation lumen.

26. A balloon catheter according to claim 25, wherein the hypotube has a distal end and the stiffness of the hypotube decreases toward said distal end.

27. A balloon catheter according to claim 25, wherein the hypotube distal end has a shaped selected from the group consisting of skived, coiled, and notched.

28. A balloon catheter according to claim 25, wherein hypotube is arranged in the inflation lumen to allow inflation fluid to pass through the half-moon-shaped inflation lumen both inside and outside of the hypotube.

29. A rapid exchange balloon catheter comprising:
a balloon segment having an expandable balloon having a proximal end and a distal end;
a shaft segment connected to the proximal end of the balloon segment, the shaft segment including an inflation lumen extending from a proximal end of the distal shaft to the interior of the balloon segment;
a guidewire tube extending through the balloon, the guidewire tube having a proximal port near the proximal end of the balloon segment and a distal port near the distal end of the balloon segment, wherein the distal guidewire port is spaced from the balloon segment a distance less than the proximal guidewire port is spaced from the balloon segment; and
a metal stiffening member positioned within a proximal portion of the inflation lumen without extending across the proximal port.

30. The balloon catheter according to claim 29, wherein the metal stiffening member comprises a tapered wire.

31. The balloon catheter according to claim 29, wherein the proximal port is positioned less than about 2 cm from the balloon segment.

32. The balloon catheter according to claim 29, wherein proximal port includes a guidewire groove having a length of about 3 cm to about 5 cm.
